(19) United States
(12) Patent Application Publication
(10) Pub. No.: US 2002/0150897 A1
(43) Pub. Date: Oct. 17, 2002

(54) Method for detecting and isolating genes

(75) Inventors: Yasuo Nagasawa, Tokyo (JP); Hideaki Yoshida, Gunma (JP)

Correspondence Address: SHANKS & HERBERT, TransPotomac Plaza, Suite 306, 1033 N. Fairfax Street, Alexandria, VA, 22314, US

(73) Assignee: Institute of Cytosignal Research, Inc.

(21) Appl. No.: 09/816,124

(22) Filed: Mar. 26, 2001

Related U.S. Application Data
(63) Continuation of application No. 09/308,164, filed on Jun. 16, 1999, now abandoned.
Application No. 09/308,164 is a 371 of international application No. PCT/JP97/04126, filed on Nov. 12, 1997.

(51) Int. Cl. C12Q 1/68; C12N 15/74; C12N 5/06; C12N 15/87

(52) U.S. Cl. 435/6; 435/455; 435/325; 435/320.1

(57) ABSTRACT

A method for detecting an inhibitory effect of a test gene on intracellular signal transduction is provided, which comprises transforming host cells by a vector carrying a gene capable of inducing cell death under specific conditions and having been ligated downstream to a promoter functioning in response to specific extracellular stimulation, introducing a test substance into the host cells or treating the host cells with the test substance so as to exracellularly stimulate the same, and then examining whether the cells survive or not to thereby detect whether the test substance has an inhibitory effect on intracellular signal transduction or not. Another detection method uses a reporter gene in place of the gene capable of inducing cell death. Furthermore, a method for isolating a gene having an inhibitory effect on intracellular signal transduction is provided, which comprises introducing a gene library into the host cells, screening surviving cells, and then isolating the introduced gene from the cells.

### TECHNICAL FIELD

The present invention relates to a method for detecting an inhibitory effect of a test substance on intracellular signal transduction and a method for isolating a gene encoding a protein that inhibits intracellular signal transduction. This invention pertains to the field of DNA cloning.

### BACKGROUND ART

Cells determine their behavior in response to extracellular information. Many kinds of signal molecules, such as growth factors, hormones, and cytokines, are responsible for intracellular signal transduction. These signal molecules transmit information to the inside of cells by binding to specific receptors on the cell membrane. In these cells, the information is transmitted to the organelles such as nuclei through substances called intracellular second messengers, resulting in expression of specific genes or like phenomena. Thus, behavior of cells is determined.

For example, expression of interleukin 8 (IL-8), one of the cytokines, is known to cause neutrophil activation. In addition, it is implied that expression of IL-8 is involved in various diseases such as inflammation (Watanebe, K. et al., Infection & Immunity 60, 1268 (1992)) and reperfusion injury in myocardial infarction or similar diseases (Sekido, N. et al., Nature 365, 654-657 (1993)). Moreover, it is implied that expression of IL-8 gene is induced through the processes of intracellular signal transduction based upon binding of tumor necrosis factor (TNF) and interleukin 1 (IL-1), which is also a kind of cytokine, to cell membrane receptors.

There are many related reports, especially on the relationship between inflammation and IL-8. IL-8 is produced in various inflammatory diseases, including chronic articular rheumatism, gouty arthritis, psoriasis, contact dermatitis, sepsis, cataplectic pulmonary fibrosis, adult respiratory distress syndrome, inflammatory enteropathy, immune angiitis, glomerulonephritis, urinary tract infection, myocardial infarction, respiratory tract infection, asthma, perinatal infection, and rejection to transplanted organs (Matsushima, K. et al., Chem. Immunol. 51,236-265 (1992)).

Since the relationship between factors involved in intracellular signal transduction and various diseases has thus been gradually clarified, factors that inhibit intracellular signal transduction have been considered for treating diseases.

However, in order to screen intracellular proteins as factors that inhibit intracellular signal transduction (specifically in the above example, in order to search for intracellular proteins that inhibit expression of IL-8 gene), a cDNA library of intracellular proteins has to be introduced into and expressed in cultured cells. I The many individual cells in which the function of producing disease inducing factors (in this case, IL-8) is thus inhibited must be screened from a huge number of cells. In such cases, there is no general method to detect individual cells which directly indicate the inhibition of production. Moreover, if IL-8 gene expression and inhibitory activity are studied by usual screening methods using cultured cells, many clones having cell toxicity are obtained. Production of IL-8 apparently decreases because substances with cell toxicity damage cells and thus inhibit spontaneous IL-8 gene expression. For example, when the above signal transduction-inhibitory activity of a chemical library and natural substances derived from microorganisms and plants is to be detected, toxic substances inhibit production of IL-8 and are thus misunderstood as signal transduction inhibitory factors (disease induction inhibitors).

Therefore, a screening method for effectively isolating a factor that inhibits intracellular signal transduction is required. There has so far been no report on positive screening methods by which cells whose intracellular signal transduction is inhibited can be distinguished at a glance.

### DISCLOSURE OF THE INVENTION

An objective of the present invention is to provide a method for detecting an inhibitory effect of a test substance on intracellular signal transduction and a method for isolating a gene encoding a protein that inhibits intracellular signal transduction.

Normal animal cells have hypoxanthine-guanine-phosphoribosyltransferase (HGPRT), and thus they can take 6-thioguanine (6-TG) into pathways of nucleic acid synthesis. If 6-TG is contained in the culture medium, the cells die because of its toxicity. However, mutant cells deficient in HGPRT cannot take 6-TG into pathways of nucleic acid synthesis and thus can live and grow in the medium containing 6-TG without being influenced by its toxicity. Therefore, the life of mutant cells deficient in HGPRT can be controlled by regulating expression of artificially introduced HGPRT (or XGPRT, which has functions similar to HGPRT) gene.

The present inventors sought to construct a system which detects a substance that inhibits intracellular signal transduction by utilizing the property of mutant cells deficient in HGPRT.

If a specific gene (gene B) is known to be expressed in response to certain extracellular stimulation (stimulation A) as an intracellular signal transduction pathway, a cell clone that activates the promoter activity of gene B in response to stimulation A to thereby express HGPRT (or XGPRT, which has the same function as HGPRT) can be prepared by selecting HGPRT-deficient cells that express gene B when stimulation A is added and introducing into the cells an expression plasmid artificially constructed by linking the promoter region of gene B to the gene encoding HGPRT (or XGPRT, which has the same function as HGPRT). In this cell clone, if stimulation A is added in the presence of 6-TG, the cells usually die because of expression of HGPRT. However, if the signal transduction pathway from stimulation A to gene B is inhibited, the cells can live because expression of HGPRT (or XGPRT) is inhibited. Therefore, it is possible to detect an inhibitory effect of a test substance on the signal transduction as mentioned above by introducing a test substance into this cell clone and confirming whether cells are alive or not after stimulation A is applied. Furthermore, the present inventors thought that, if a gene library is introduced into this cell clone, it is theoretically possible to effectively isolate a gene that inhibits the signal transduction described above by selecting cells that are alive after stimulation A is applied.

Under these circumstances, the present inventors studied how to detect and isolate factors that inhibit intracellular signal transduction based upon the principle above. In particular, the present inventors thought that substances with the inhibitory effect on TNF and IL-1 as well as those that inhibited expression of IL-8 could also be detected by screening with the inhibition of IL-8 production as a marker because various diseases including inflammation accompany the production of TNF (Molecular Medicine 33 1010-1020 (1996)), IL-1 (Clin. Immunol. 27 18-28 (1995)), and IL-8 (Clin. Immunol. 27 80-85 (1995)) and because the production of IL-8 is induced by TNF and IL-1 (Mukaida, N. et al. Microbiol. Immunol. 36 773 (1992)). Therefore, the present inventors studied a method for selecting the pathway through which IL-8 is expressed in response to TNF stimulation as an intracellular signal transduction pathway and for detecting and isolating a gene encoding a protein that inhibits the process from binding TNF to its receptor to the production of IL-8 through the intracellular signal transduction.

The present inventors first, isolated the gpt gene encoding XGPRT, which has almost the same function as HGPRT, and promoter of human IL-8 by PCR, and constructed a plasmid vector with the gpt gene downstream of the IL-8 promoter. The present inventors then introduced said vector into HGPRT-deficient cells and selected clones that induce cell death by adding TNF stimulation. The thus-obtained cell clones, when activated intracellular IL-8 promoter by means of TNF stimulation, expressed the gpt gene downstream to the promoter, and died if 6-TG was added to the culture medium. Furthermore, it has been reported that expression of IL-8 is observed generally in inflammation (Watanabe, K. et al., Infection & Immunity 60, 1268 (1992), Matsushima, K. et al., Chem. Immunol. 51, 236-265 (1992)) and that inflammation is inhibited when the function of IL-8 is inhibited by an anti-IL-8 antibody (Harada, A. et al., Int. Immunol. 5, 681-690 (1993), Sekido, N. et al., Nature 365, 654-657 (1993)). In addition, it has been reported that dexamethasone, which is known as an anti-inflammatory steroid, has an inhibitory effect on activation of the IL-8 promoter (Mukaida, N. et al., J. Immunol. 146, 1212-1215 (1991), Mukaida, N. J., Biol. Chem. 269, 13289 (1994)). Therefore, the present inventors expected that treatment with dexamethasone of the isolated clone would suppress activation with TNF of the IL-8 promoter linked with the gpt gene. In consistence with this thought, cell death of the clone by TNF stimulation was markedly inhibited by this treatment with dexamethasone.

Thus, a cell clone which is killed when IL-8 promoter is activated by TNF but survives with the existence of an anti-inflammatory steroid has been successfully developed. The present inventors thought that this cell clone could be used as a system to assess cDNA which encodes an anti-inflammatory protein that is involved in inhibiting signal transduction from TNF stimulation up to the activation of IL-8 promoter.

The present inventors then introduced a cDNA library into the isolated clone and performed the first screening of cDNA involved in inhibiting signal transduction from TNF stimulation to the activation of IL-8 promoter. From this screening, the present inventors obtained several effective cDNAs.

The present inventors then performed a second screening for the cDNAs obtained in the first screening in order to confirm the inhibitory effect on the IL-8 promoter. The second screening was performed using a luciferase expression plasmid having IL-8 promoter as a reporter gene.

A recombinant expression vector with the cDNA obtained in the first screening was cortransfected with the above luciferase expression plasmid. This established a system in which expression of the luciferase gene downstream of the IL-8 promoter was inhibited if the introduced cDNA could inhibit the signal transduction from TNF stimulation to activation of the IL-8 promoter, and, conversely, the expression of luciferase was fully promoted unless the introduced cDNA inhibited the signal transduction. Using this system, luciferase activity was measured for every cDNA to be tested. Furthermore, the vector without the cDNA was used as the negative control. As a result, the present inventors found that luciferase activity was strongly induced (IL-8 promoter was activated) with TNF in the negative control without cDNA whereas activation of IL-8 promoter was inhibited for four out of six test cDNAs as in the dexamethasone treatment.

Thus, the present inventors proved that the cloning system using the gpt gene and the HGPRT-deficient cells was capable of finding cDNAs involved in inhibiting intracellular signal transduction. Furthermore, the cloning system was used as the expression cloning system to obtain the above cDNAs efficiently, thus completing the present invention.

More specifically, the present invention relates to

(1) a vector holding a gene capable of inducing cell death under specific conditions and linked downstream to a promoter region that functions in response to specific extracellular stimulation,

(2) the vector of (1), wherein said extracellular stimulation is stimulation by cytokine,

(3) the vector of (1), wherein said extracellular stimulation is stimulation by tumor necrosis factor (TNF),

(4) the vector of (3), wherein said promoter region functioning in response to specific extracellular stimulation is a promoter region of interleukin 8 gene,

(5) the vector of any one of (1) to (4), wherein said gene inducing cell death under specific conditions is xanthine-guanine-phosphoribosyltransferase (gpt) gene,

(6) a host cell transformed with the vector of any one of (1) to (5),

(7) the host cell of (6), wherein said cell cannot produce hypoxanthine-guanine-phosphoribosyltransferase (HGPRT), and said gene inducing cell death under specific conditions in the vector to be inserted is xanthine-guanine-phosphoribosyltransferase,

(8) a method for detecting an inhibitory effect of a test substance on intracellular signal transduction, wherein the method comprises,

(a) introducing the test substance into or allowing the substance to act on the cells of (6),

(b) testing whether the cells obtained in (a) that the test substance is introduced into or acts on are alive or not when the specific extracellular stimulation which causes said specific intracellular signal transduction under the specific conditions is added to the cells,

(9) a method for detecting an inhibitory effect of a test substance on intracellular signal transduction, wherein the method comprises,

(a) introducing the test substance into or allowing the substance to act on the cells of (7),

(b) testing whether the cells that the test substance is introduced into or acts on obtained in (a) are alive or not when the specific extracellular stimulation which causes said specific intracellular signal transduction is added to the cells in the presence of 6-thioguanine,

(10) the method of (8) or (9), wherein said test substance is a gene,

(11) the method of (8) or (9), wherein said test substance is a low molecular weight compound,

(12) a method for isolating a gene encoding a protein that inhibits specific intracellular signal transduction, wherein the method comprises,

(a) introducing a gene library into the cells of (6),

(b) screening living cells after the specific extracellular stimulation which causes said specific intracellular signal transduction is added under the specific conditions to the cells that the gene library is introduced into obtained in (a),

(c) isolating the gene introduced into said cells from the cells screened in (b),

(13) a method for isolating a gene encoding a protein that inhibits specific intracellular signal transduction, wherein the method comprises,

(a) introducing a gene library into the cells of (7),

(b) screening living cells after the specific extracellular stimulation which causes said specific intracellular signal transduction is added in the presence of 6-thioguanine to the cells that the gene library is introduced into obtained in (a),

(c) isolating the gene in said cells from the cells screened in (b),

(14) the method of any one of (8) to (13), wherein said extracellular stimulation is stimulation by cytokine,

(15) the method of any one of (8) to (13), wherein said extracellular stimulation is stimulation by tumor necrosis factor (TNF),

(16) the method of (15), wherein said promoter region functioning in response to specific extracellular stimulation is a promoter region of interleukin 8 gene,

(17) the method of any one of (8) to (13), wherein said vector introduced into the cells of (6) or (7) is the vector of (4),

(18) a method for detecting an inhibitory effect of a test gene on intracellular signal transduction, wherein the method comprises,

(a) introducing into host cells a vector comprising a test gene that can be expressed in the host cells and a vector having a reporter gene downstream to a promoter region functioning in response to specific extracellular stimulation,

(b) applying specific extracellular stimulation to the host cells obtained in (a) into which the vector is introduced and detecting the activity of a reporter gene product,

(19) a method for detecting an inhibitory effect of a low molecular weight compound to be tested for intracellular signal transduction, wherein the method comprises,

(a) introuducing into host cells a vector having a reporter gene downstream to a promoter region functioning in response to specific extracellular stimulation,

(b) allowing a low molecular weight compound to be tested to act on the host cells into which the vector obtained in (a) is introduced and detecting the activity of the reporter gene product,

(20) a method for isolating a gene encoding a protein that inhibits specific intracellular signal transduction, wherein the method comprises,

(a) introducing into host cells a gene library that can be expressed in the host cells and a vector having a reporter gene downstream to a promoter region functioning in response to specific extracellular stimulation,

(b) applying specific extracellular stimulation to the host cells into which the vector in (a) is introduced, detecting the activity of the reporter gene product, and selecting cells in which said activity decreases,

(c) isolating a gene introduced into said cells from the cells screened in (b),

(21) the method of any one of (18) to (20), wherein said reporter gene is the luciferase gene,

(22) the method of any one of (18) to (21), wherein said extracellular stimulation is stimulation by cytokine,

(23) the method of any one of (18) to (21), wherein said extracellular stimulation is stimulation by tumor necrosis factor (TNF),

(24) the method of (23), wherein said promoter region functioning in response to specific extracellular stimulation is a promoter region of the interleukin 8 gene,

(25) a host cell transformed with a vector comprising a test gene that can be expressed in the host cells and a vector having a reporter gene downstream to a promoter region functioning in response to specific extracellular stimulation,

(26) the host cells of (25), wherein said extracellular stimulation is stimulation by cytokine,

(27) the host cells of (25), wherein said extracellular stimulation is stimulation by tumor necrosis factor (TNF),

(28) the host cells of (27), wherein said promoter region functioning in response to specific extracellular stimulation is a promoter region of interleukin 8 gene,

(29) the host cells of any one of (25) to (28), wherein said reporter gene is luciferase gene.

The present invention first relates to a method for detecting an inhibitory effect of a test substance on intracellular signal transduction. One indication method uses the life of cells and the other uses a reporter gene.

In the first stage of the method in which detection is performed using the life of cells as an indication, a vector in which a gene inducing cell death under specific conditions is linked downstream to a promoter region functioning in response to specific extracellular stimulation is constructed, and host cells are transformed with the vector.

There is no limitation on the promoter region functioning in response to specific extracellular stimulation. For example, the promoter region of the IL-8 gene can be used as the functional promoter region when TNF, IL-1, lipopolysaccharide (LPS), phorbol myristate acetate (PMA), or similar extracellular stimulating factors is used, and the promoter region of the IL-6 and COX-2 gene can be used as the promoter region when TNF, IL-1, LPS, or PMA is used as the extracellular stimulating factor. Moreover, the promoter region of the IL-1 gene can be used as the promoter region if TNF, LPS, or PMA is used as the extracellular stimulating factor. The promoter region of the TNF gene can be used as the promoter region if IL-1, LPS, or PMA is used as the extracellular stimulating factor. A gene encoding an enzyme that converts some compound into some toxic substance can be used as a tool inducing cell death under specific conditions. Examples thereof include the gpt gene, HSV-tk (Herpes Simplex Virus thymidine kinase) gene, hypoxanthine-phosphoribosyltransferase gene, VZV-tk (Varicella Zoster Virus thymidine kinase) gene, and cytosine deaminase gene, but are not limited to these genes.

Specific conditions are the presence of a substance taken into pathways of nucleic acid synthesis instead of guanine to show toxicity, for example, purine analogue such as 6-TG and 8-azaguanine (8-AG) if the gpt gene is used. The substances taken into pathways of nucleic acid synthesis to show toxicity include, for example, ganciclovir and fluoroiodoadenosyluracil (FIAU) if the HSV-tk gene is used, 6-TG or 8-TG if hypoxanthine-phosphoribosyltransferase gene is used, 6-methoxypurinearabinonucleoside if the VZV-tk (Varicella Zoster Virus thymidine kinase) gene is used, and 5-fluorocytosine if the cytosine deaminase gene is used. However, the conditions are not limited thereto.

Furthermore, a gene encoding a toxic protein, for example, ricin (toxic protein from castor-oil plant seeds), abrin (toxic protein from jequirity seeds), diphtheria toxin, and cholera toxin can be used as the gene inducing cell death. In this case, they do not need particular conditions as required for the genes described previously since expression of these genes alone induces cell death. Host cells vary depending on the gene inducing cell death. For example, HGPRT-deficient cells are highly preferable if the gpt gene is used. There is no limitation for cells used if the HSV-tk gene is used. In this case, human VA-13 cells, human RERF-LC-AI cells (Riken Cell Bank), and so on are preferable.

If the HGPRT (or XGPRT) gene is used, HGPRT-deficient cells have to be prepared as the host cells to be transformed. However, if a gene originally absent in host cells, such as those encoding cytosine deaminase, ricin, abrin, diphtheria toxin, or cholera toxin, is used, cells lacking these genes do not have to be prepared. In addition, if the HSV-tk gene or the VZV-tk gene is used, cells lacking the gene do not have to be prepared because the specificity of these thymidine kinases to drugs is different from the thymidene kinase possessed by usual host cells.

HGPRT-deficient cells can be prepared, for example, by cultivating cells in the presence of purine analogue such as 6-TG or 8-AG and by isolating cells which grow in the presence of these drugs and which exhibit a cell growth inhibitory effect (Littlefield, J. W. Proc. Natl. Acad. Sci. USA. 50, 568 (1963)).

A promoter region functioning in response to specific extracellular stimulation and a gene inducing cell death can be isolated as follows if all or part of the nucleotide sequence of a desired gene or the amino acid sequence of a desired protein is known. The sense and antisense strand oligonucleotides corresponding to the part of the gene or the amino acid sequence are synthesized. cDNA is synthesized from mRNA of cells expressing a desired protein through reverse transcription. Polymerase chain reaction (Saiki, R. K. et al., Science 239, 487-491 (1988)) is performed with these oligonucleotides as the primers and with the cDNA or genomic DNA as the template to amplify the desired gene. The thus-prepared DNA fragments are labeled with ³²P or 35S, and used as probes for colony hybridization or plaque hybridization to select a desired clone.

A vector in which a gene inducing cell death under specific conditions is linked downstream to the promoter region can be constructed by commonly used gene manipulation as described in “Molecular Cloning” (Sambrook, J. et al., Molecular Cloning: A Laboratory Manual, 2nd ed. Cold Spring Harbor Laboratory, NY (1989)) or “Laboratory Manual: Gene Engineering” (ed. Muramatsu, M. Maruzen (1990)). There is no limitation on the vectors into which said gene is inserted. For example, vectors with the E. coli replication origin and the ampicillin resistance gene, which are necessary for replication in E. coli, and with a unit for expressing a selection marker, which is necessary for obtaining stable transfectants in host animal cells, are preferable. Specifically, vectors such as pMAM-neo (Toyobo, CLONETECH) and pREP9 (Funakoshi, INVITROGEN) can be used.

Vectors can be introduced into host cells by the DEAE-dextran method (Luthman, H. et al., Nucleic Acids Res. 11, 1295-1308 (1983)), calcium phosphate method (Graham, F. L. et al., Virology 52, 456-457 (1973)), electroporation method (Neumann, E. et al., EMBO J. 1, 841-845 (1982)), and so on.

Host cells thus obtained die under the specific conditions if the promoter on the vector introduced into the cells is activated upon specific extracellular stimulation and the downstream gene inducing cell death is thus expressed.

In the second stage of the detecting method in the present invention, a test substance is introduced into or allowed to act on the host cells into which the above-described vector is introduced, extracellular stimulation is applied under specific conditions, and whether the host cells are alive or not is examined.

There is no limitation on the test substance. When a gene is used as a test substance, it is inserted into an appropriate expression vector and introduced into host cells. There is no limitation on the expression vector. Preferable examples of the vector are derivatives of pcDL-SRα296 (Takebe, Y. et al., Mol. Cell. Biol. 8, 466-472 (1988)), which has the SRα promoter that is capable of expressing a test gene efficiently, and derivatives of pEF-BOS (Mizushima, S. Nucleic Acids Res. 18, (1990)), which has a promoter of the elongation factor.

The gene to be tested can be introduced into host cells by the DEAE-dextran method (Luthman, H. et al., Nucleic Acids Res. 11, 1295-1308 (1983)), calcium phosphate method (Graham, F. L. et al., Virology 52, 456-457 (1973)), electroporation method (Neumann, E. et al., EMBO J. 1, 841-845 (1982)), and so on.

Moreover, a compound other than genes can be used as a test substance. There is no limitation on these compounds. Natural or synthesized low molecular weight compounds can be used. A culture supernatant of specific microorganisms can also be used. Low molecular weight compounds can act by simply adding them to the culture medium.

There is no limitation on the method by which the extracellular stimulating factors are applied to host cells. This can be done by simply adding them to the culture medium. When TNF is used as the extracellular stimulating factor, the amount to be added is usually 1 to 1000 U/ml (0.05 to 50 ng/ml) and desirably 20 to 100 U/ml (1 to 5 ng/ml).

Whether cells are alive or dead is judged by observation under a microscope; quantitative measurement of the amount of the reduction product of Alamar Blue taken into cultured cells in terms of fluorescence intensity or absorbance; quantitative measurement of cell growth; reduction of MTT (J. Immun. Methods 65, 55-63 (1983)); measurement of intake of ³H-thymidine or pigment such as Neutral Red; dye-exclusion test using, for example, Trypan Blue; and so on.

If the cells survive, the test substance introduced into the cells has an inhibitory effect on intracellular signal transduction. In contrast, if the cells die, the test substance introduced into the cells has no inhibitory effect on intracellular signal transduction.

If this judgement reveals that cells survive when a culture supernatant of specific microorganisms is used as a test substance, a compound having the inhibitory effect on intracellular signal transduction can be purified from this supernatant by fractionating it by column chromatography and repeating the detecting method of the present invention for the resulting fractions.

The above-described method for detecting the inhibitory effect of a test substance on intracellular signal transduction can be applied to isolating a gene encoding a protein that inhibits intracellular signal transduction (first screening method). Surviving cells are screened after extracellular stimulation is applied using a gene library instead of a specific test substance. A gene introduced into the cells is isolated. The isolated gene is a candidate for a gene encoding a protein that inhibits intracellular signal transduction.

The gene library to be used can be constructed by the Gubler-Hoffmann method (Gubler, U. et al., Gene 25, 263-269 (1983)) and so on.

In addition, a gene introduced into cells can be isolated by PCR amplification and recovering the introduced plasmid DNA including cDNA using the genomic DNA as the template, the plasmid rescue method (Yokota et al., Experimental Method for Gene Cloning, YODOSHA (1993)), and so on.

The other detecting method of the present invention relates to detecting a reporter gene as a marker. In this method, a vector comprising a test gene and a vector having a reporter gene downstream of the promoter region responding to specific extracellular stimulation are introduced into host cells, specific extracellular stimulation is applied to the cells, and the activity of the reporter gene product is detected.

For example, a vector having the luciferase gene downstream to the promoter region can be used. The vector can be constructed by commonly used gene manipulation as described in “Molecular Cloning” (Sambrook, J. et al., Molecular Cloning: A Laboratory Manual, 2nd ed. Cold Spring Harbor Laboratory, NY (1989)) or “Laboratory Manual: Gene Engineering” (ed. Muramatsu, M. Maruzen (1990)). For example, pGL2-Promoter Vector (Promega) can be used as the vector into which a promoter region and a reporter gene are inserted.

These vectors can be introduced into host cells by the DEAE-dextran method (Luthman, H. et al., Nucleic Acids Res. 11, 1295-1308 (1983)), calcium phosphate method (Graham, F. L. et al., Virology 52, 456-457 (1973)), electroporation method (Neumann, E. et al., EMBO J 1, 841-845 (1982)), and so on. Preferable examples of the host cells include MRC-5 SV1 TG1 cells (Riken Cell Bank), VA-13 cells (Riken Cell Bank), and RERF-LC-AI cells (Riken Cell Bank).

The activity of luciferase can be measured, for example, by the method of Williams et al. (Williams, T. M. et al., Anal. Biochem. 176, 28-32 (1989)).

Genes other than the luciferase gene as described above, such as genes encoding CAT (chloramphenicol acetyltransferase) (Gorman, C. M. et al., Mol. Cell. Biol. 2, 1044-1051 (1982)), β-galactosidase (Jain, V. et al., Anal. Biochem. 199, 119-124 (1991)), β-glucuronidase (Gallagher, S. R. GUS Protocol: Using the GUS as a Reporter of Gene Expression, Academic Press, 47-59 (1992)), and alkaline phosphatase (Cullen, B. et al., Methods in Enzymology 216, 362-368 (1992)) can be used as the reporter gene.

The luciferase activity inhibiting effect of a test gene can be compared with that obtained by using a vector with no such a gene as a control.

As a result, a gene showing the luciferase activity inhibiting effect is effectively confirmed to be the desired gene which suppresses intracellular signal transduction.

It is possible to determine whether or not the test substance has the intracellular signal transduction-inhibitory effect, by allowing a test substance other than genes, for example, a low molecular weight compound, to act on host cells instead of introducing a vector with a test gene introduced into the host cells and detecting the activity of the reporter gene.

This is the preferable method for isolating a gene encoding a protein that suppresses intracellular signal transduction (however, positive screening is impossible). In this detection method, a gene that suppresses intracellular signal transduction can be isolated by introducing a gene library instead of a specific gene to be tested, detecting the activity of the reporter gene after extracellular stimulation is applied to host cells, and selecting clones with a low level of the activity. This isolating method is especially effective as the second screening method after the first screening method described above.

### BRIEF DESCRIPTION OF THE DRAWINGS

FIG. 1 shows the structure of pREP9-IL8p-gpt-neo.

FIG. 2 shows the sequence of DNA primers for constructing pBlue-SR.

FIG. 3 shows the MCS template DNA sequence.

FIG. 4 shows the process of constructing pBlue-SR, in which DNA fragments comprising four regions of SRα promoter (Fragment A), splicing site (Fragment B), multiple cloning site (MCS) (Fragment C), and polyadenylation signal (Fragment D) are ligated.

FIG. 5 shows the process of constructing pBlue-SR, in which the DNA fragments comprising the four regions are inserted into the vector.

FIG. 6 shows the sequence of synthetic DNA primers for constructing pBlue-SRα-Hind.

FIG. 7 shows the sequence of the inserted DNA on pBlue-SRα-Hind.

FIG. 8 shows the process of constructing pBlue-SRα-Hind.

FIG. 9 shows the process of constructing pBlue-SRα-lacO.

FIG. 10 shows the process of constructing pBlue-SRO.

FIG. 11 shows the process of constructing pBlue-SROL.

FIG. 12 shows the process of constructing pSROL-3′SS(NB2).

FIG. 13 shows the structure of pSRO-cDNA.

FIG. 14 shows the structure of the reporter plasmid vector pIL8p-Luc.

FIG. 15 shows the result of examining the living cell measurement method using Alamar Blue.

FIG. 16 shows the effect of an extracellular stimulating factor (TNF-α) and dexamethasone treatment on the growth of cell clone IL8p-gpt-neo#17.

FIG. 17 shows the PCR primers for extracting cDNA derived from the pSRO-cDNA library integrated into genomic DNA of the cells.

FIG. 18 shows the result of detecting IL-8 promoter inhibitory activity of cDNA isolated by the screening method of the present invention.

### BEST MODE FOR IMPLEMENTING THE INVENTION

### EXAMPLE 1

### Construction of Plasmid pREP9-IL8p-gpt-neo (abbreviated pIL8p-gpt-neo) having the gpt Gene Downstream to the IL-8 Promoter and the Neomycin Resistance Gene, Neo

(1) Construction of pBluescript SK(+)-gpt

About 600 bp of the gpt gene region was amplified by PCR using Taq polymerase (TAKARA), the following primers, and pSV2-gpt (Mulligan, R. C. and Berg, P. Science 209, 1422-1427 (1980)) as the template.

| | |
|---|---|
| Primer #1 | |
| ATAAGCTTTTCACATGAGCGAAAAATACA | (SEQ ID NO: 1) |
| HindIII | |
| Primer #2 | |
| ATGGATCCCTATTGTAACCCGCCTGAAGT | (SEQ ID NO: 2) |
| BamHI | |

PCR was performed with DNA Thermal Cycler Model PJ2000 (TAKARA, PERKIN ELMER CETUS). The reaction mixture contained a PCR buffer (10 mM Tris-HCl, pH 8.3, 50 mM KCl, 1.5 mM MgCl₂, 0.001% gelatin), 0.2 mM each (final concentration) of dNTPs (dATP, dCTP, dGTP, dTTP), 1 μM DNA primers, and the template DNA to make the total volume 100 μl. Twenty cycles of incubation at 94° C. for 1 minute, at 50° C. for 1 minute, and at 72° C. for 1 minute were performed. The PCR product was digested with restriction enzymes BamHI and HindIII and purified with the DNA purifying agent preA-gene Matrix (Nippon Bio-Rad Laboratories, BIO-RAD). The fragment was ligated into BamHI- and HindIII-digested pBluescript II SK(+) (TOYOBO, STRATAGENE) with a DNA Ligation Kit (TAKARA) using T4 DNA ligase. Colonies resistant to ampicillin were obtained by introducing the resulting plasmid into the competent cells of XL1-Blue, an E. coli K-12 strain. The plasmids into which the gpt gene was inserted were obtained by collecting plasmids from cultured cells of colonies and examining the restriction pattern. DNA sequencing was performed for the part corresponding to the coding sequence of the gpt gene, and the sequence was confirmed to be the desired gpt gene. DNA sequencing was performed with Taq DyeDeoxy ™Termination Cycle Sequencing Kit (APPLIED BIOSYSTEMS) along with its protocol. After the reaction, the reaction product was purified with a spin column Bio-Spin 30 (BIO-RAD) and analyzed with a DNA sequencer (ABI 373A DNA Sequencing System).

(2) Construction of pREP-gpt

The BamHI-HindIII fragment (about 600 bp) of pBluescript SK(+)-gpt containing the gpt gene and the BamHI-HindIII fragment—(about 10 kb) of pREP9 (FUNAKOSHI, INVITROGEN) were recovered by low-melting point agarose gel electrophoresis and purified with a tip for isolating and purifying nucleic acid, QIAGEN-tip 5 (FUNAKOSHI, QIAGEN Inc.). The two fragments were ligated to each other with DNA Ligation Kit (TAKARA) then introduced into competent cells of E. coli K-12 strain XL1-Blue to obtain colonies resistant to ampicillin. Plasmid DNAs of the transformants were prepdred, and clones with the gpt gene were selected. The plasmid thus obtained was named pREP-gpt.

(3) Construction of pREP-IL8-gpt-neo

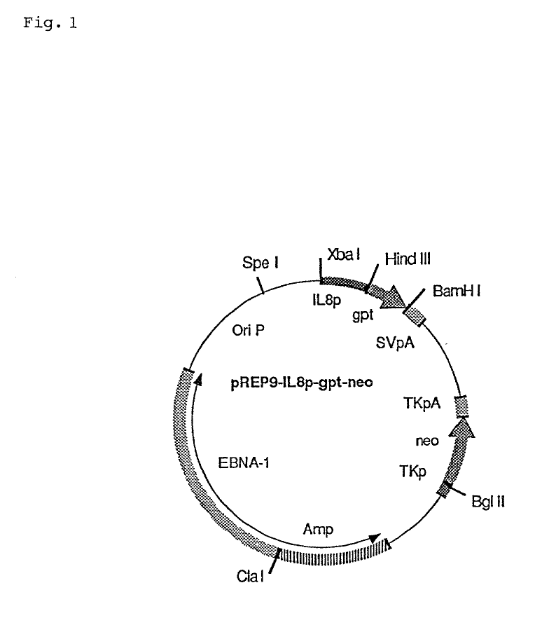

The human IL-8 promoter region was introduced into pREP-gpt obtained above so that expression of the gpt gene could be regulated. More specifically, DNA fragments containing the human IL-8 (neutrophil chemotactic cytokine) promoter region (Matsushima, K. et al., J. Imm. 143, 1366-1371 (1989)) were prepared by PCR with synthetic primers IL8P1 (5′-ATGTCTCGAGAATTCAGTAACCCAGGCATTATTTTATC-3′ (SEQ ID NO:3)) and IL8P2 (5′-TTGTCCTAGAAGCTTGTGTGCTCTGCTGTC-3′ (SEQ ID NO:4)), and with a genomic DNA of human VA-13 cells (Riken Cell Bank) as the template. After the fragments were digested with HincII and HindIII, the human IL-8 promoter region (IL8p, −546 to +44) was recovered and purified. pREP-gpt was digested with XbaI, blunted with a DNA Blunting Kit (TAKARA), digested with HindIII, and a 10 kb DNA fragment was purified. The two fragments were ligated with a DNA Ligation Kit (TAKARA) then introduced into the competent cells of JM109 (TOYOBO) to obtain colonies resistant to ampicillin. The plasmid with the structure shown in FIG. 1 was obtained by collecting plasmids from cultured cells of colonies and examining the restriction pattern. In this plasmid, the E. coli gpt gene was ligated to the downstream end (3′-end) of the human IL-8 promoter region (IL8p, −546 to +44), and xanthine-guanine-phosphoribosyltransferase (XGPRT) was thus produced under the control of the human IL-8 promoter. In addition, this plasmid had the neomycin resistance gene, neo, derived from pREP9, as the selection marker.

### EXAMPLE 2

### Construction of Expression Vector for cDNA Library Construction

(1) Construction of pBlue-SR

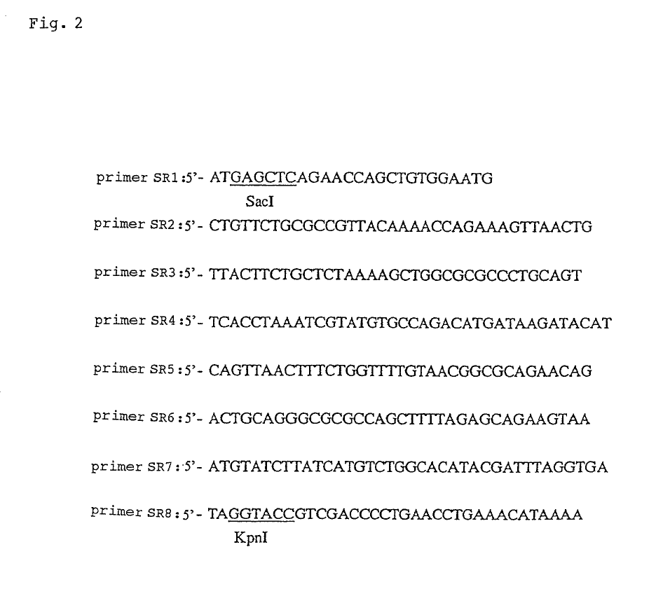

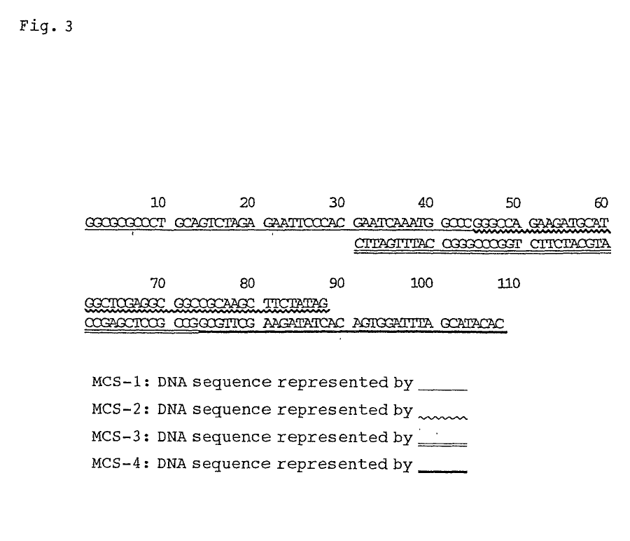

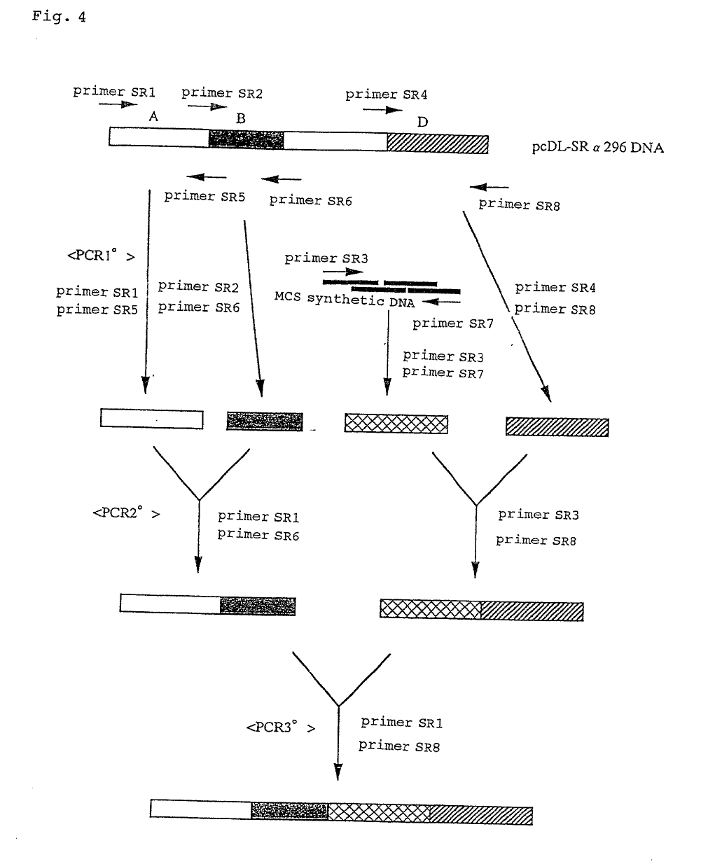

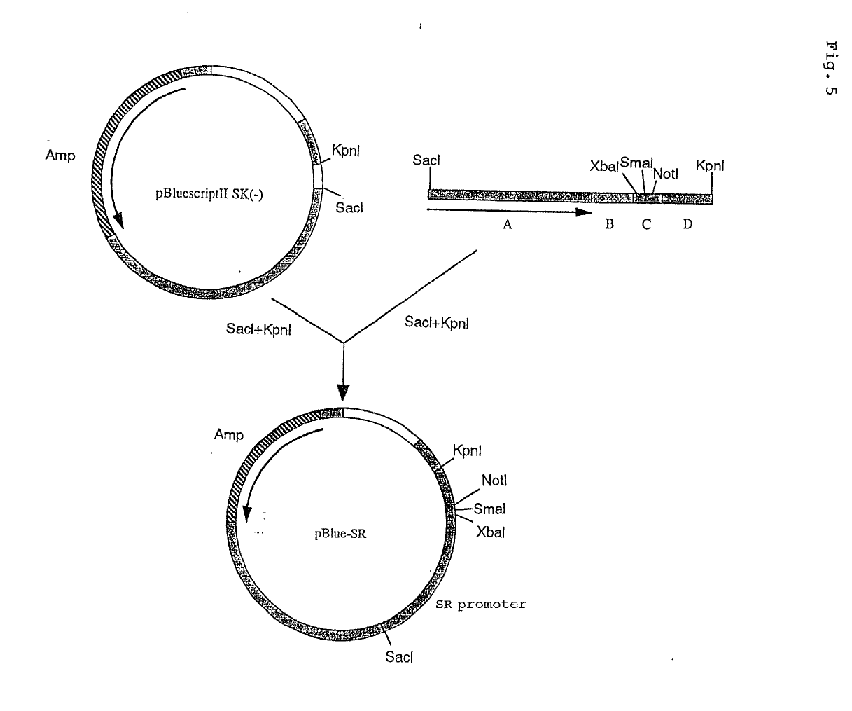

A vector that utilizes pcDL-SRα296 (Takebe Y. Mol. Cell. Biol. 8, 466-472 (1988)) as the DNA region necessary for expression in animal cells was constructed. pcDL-SRα296 is known for highly efficient expression in various kinds of cultured cells. The unit for driving transcription was reconstructed by the gene fusion method using PCR (Vallete, F. et al., Nucleic Acids Res. 17, 723-733 (1989)). After the SRα promoter (Fragment A), splicing site (Fragment B), multiple cloning site (MCS) (Fragment C), and polyadenylation signal (Fragment D) were prepared by PCR, these fragments were fused. More specifically, the first PCR was performed using the synthetic DNAs shown in FIG. 2 as primers, the annealing product of the four synthetic DNAs shown in FIG. 3 as the template for the multiple cloning site (MCS), and pcDL-SRα296 as the template for the other regions. FIG. 4 shows the process of linking DNA fragments containing the four regions by PCR. Since the DNA primers used for linking each DNA fragment were designed to have the sequence of the DNA to be amplified at its 3′ end and have the sequence of the adjacent DNA to be connected at its 5′ end, four amplified DNA fragments (A, B, C, and D) have overlapping parts (30 to 40 bp) between two adjacent regions. These DNA fragments were separated by low-melting point agarose gel electrophoresis and purified with a tip for isolating and purifying nucleic acid, QIAGEN-tip 5 (FUNAKOSHI, QIAGEN Inc.). After equal volumes of Fragments A and B were mixed, Fragment AB was prepared by PCR with outside primers (Primers SR1 and SR6). Fragment CD was prepared through a similar process with Primers SR3 and SR8. Finally, Fragment ABCD (about 1 kb) was prepared with equal volumes of purified Fragments AB and CD by PCR with outside primers (Primers SR1 and SR8). The resulting Fragment ABCD was digested with SacI and KpnI whose sites were made outermost in said fragments to purify and recover about 1 kb SacI-KpnI Fragment ABCD. The Fragment ABCD was ligated with about 3 kb SacI-KpnI fragment of pBluescript II SK(-) (TOYOBO, STRATAGENE). The ligation product was introduced into E. coli and cloned. FIG. 5 shows the plasmid DNA pBlue-SR thus obtained

(2) Construction of pSROL

To apply the Lac repressor system that was reported to enable inducible expression in animal cells (Mickey, C.-T. Cell 48, 555-566 (1987)), a vector plasmid, pSROL, was constructed by modifying pBlue-SR.

2-1) Construction of pBlue-SRα-Hind

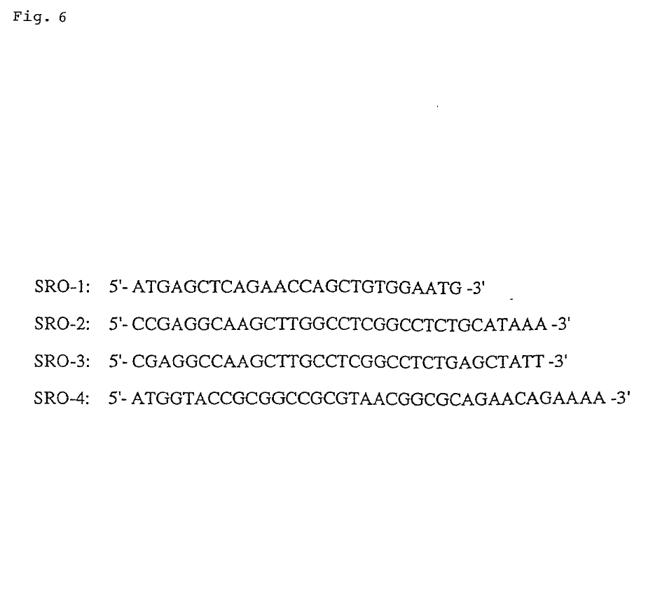

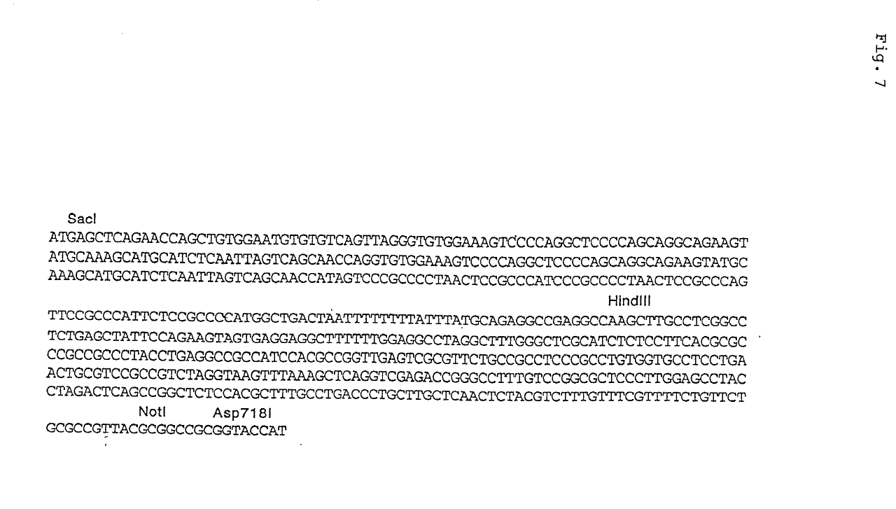

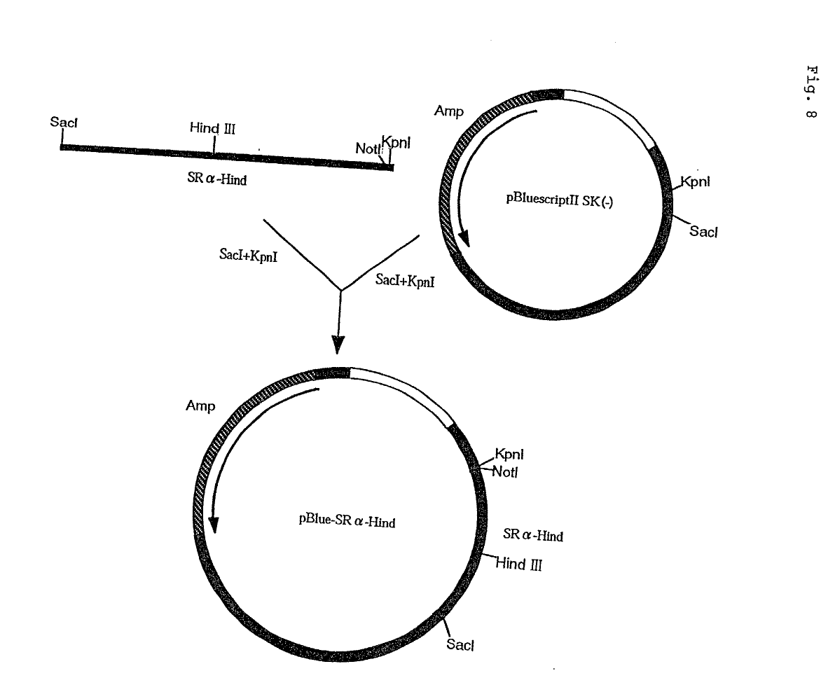

The position of Lac operator sequence between a promoter and a gene is thought to be important for regulating expression with Lac repressor (Brown, M. Cell 49, 603-612 (1987)). SRα promoter consists of SV40 early promoter fused with the LTR of HTLV-1 (Mol. Cell. Biol. (1988) 466-472). A HindIII site was introduced immediately before the transcription initiation site within the SV40 early promoter region, which exists at the upstream site of the junction between two units of SRα promoter, by mutagenesis using PCR (Higuchi, R. PCR Protocols, Academic Press, Inc. 177-183 (1990)) in order to insert the Lac operator sequence into this HindIII site. An SRα promoter fragment with a HindIII site was prepared using the synthetic DNAs shown in FIG. 6 as the primers and pBlue-SRα as the template. FIG. 7 shows the sequence of the prepared DNA. The detailed process of the construction is described below. The first PCR was performed with the primer pair, SRO-1 and SRO-2 (or SRO-3 and SRO-4) using the pBlue-SRα plasmid DNA as the template. Each PCR product was recovered by low-melting point agarose gel electrophoresis and purified with a tip for isolating and purifying nucleic acid, QIAGEN-tip 5 (FUNAKOSHI, QIAGEN Inc.). The reaction mixture contained a PCR buffer (10 mM Tris-HCl, pH 8.3, 50 mM KCl, 1.5 mM MgCl₂, 0.001% gelatin), and 0.2 mM each (final concentration) of dNTPs (dATP, dCTP, dGTP, and dTTP). Twenty cycles of incubation at 94° C. for 1 minute, 55° C. for 1 minute, and 72° C. for 1 minute were performed. In the second PCR, after two cycles were performed for a mixture with equal amounts of the two DNA fragments amplified in the first PCR, additional 18 cycles were performed with Primers SRO-1 and SRO-4 under the same conditions as described above. The resulting PCR product was digested with restriction enzymes SacI and KpnI, purified, and subcloned into pBluescript II SK(-) (FUNAKOSHI) which had been digested with the same restriction enzymes. The thus obtained clone plasmid was named pBlue-SRα-Hind (FIG. 8), and its nucleotide sequence was determined.

2-2) Construction of pBlue-SRα-lacO

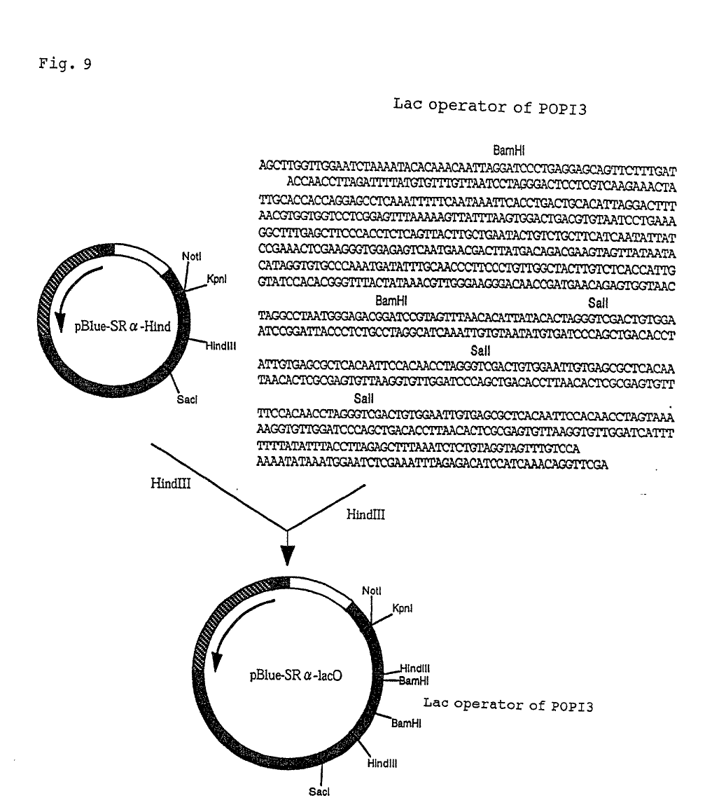

The plasmid obtained in 1) above was digested with HindIII, and a DNA fragment (about 3.5 kb) was recovered. Separately, pOP13 (TOYOBO, STRATAGENE) was also digested with HindIII to recover a DNA fragment (about 500 bp). This DNA fragment contains three Lac operator sequences. These two fragments were ligated, and the competent cells of E. coli K-12 strain, XL1-Blue, were transformed with the ligation product. The thus-obtained colonies were cultivated, and isolated plasmids were analyzed. Thus, pBlue-SR α-lacO, in which the operator was inserted in the same direction as SRα promoter, has been constructed (FIG. 9).

(3) Construction of pBlue-SRO

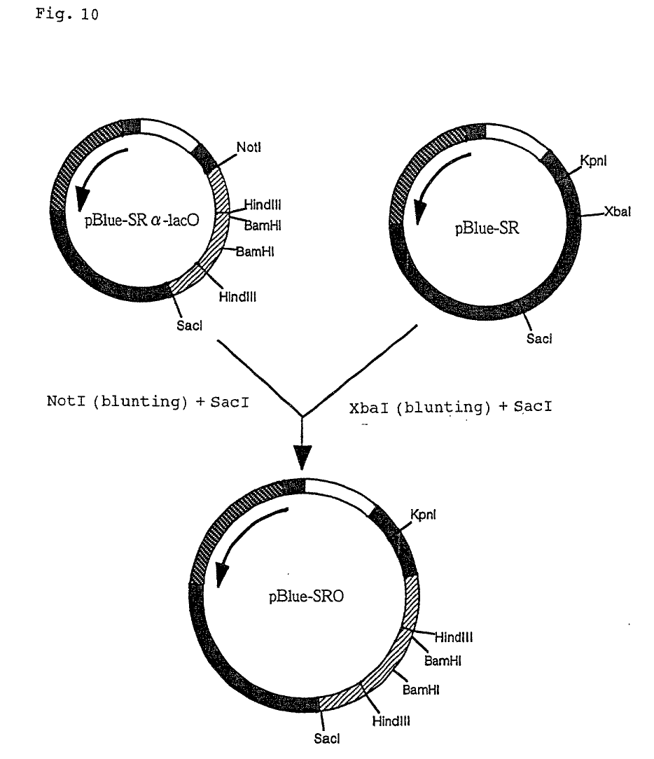

pBlue-SRα-lacO was digested with NotI, blunted, and digested with SacI to obtain a DNA fragment (about 1 kb). Separately, pBlue-SR was digested with SacI and XbaI (for blunting) to obtain a DNA fragment (about 3.2 kb). These two fragments were ligated to each other. By analyzing the clones obtained by transformation with the ligated fragments, pBlue-SRO was constructed (FIG. 10).

(4) Construction of pBlue-SROL

4-1) Construction of pBlue-Luc

pGV-CS (TOYO INK), a cassette vector of the luciferase gene, was digested with Xho I, blunted, and digested with BamHI. Separately, pBluescript II SK(+) (TOYOBO, STRATAGENE) was digested with XbaI (for blunting) and digested with BamHI. By ligating these two DNA fragments, plasmid pBlue-Luc, in which the luciferase gene was subcloned, was constructed.

4-2) Construction of pBlue-SROL

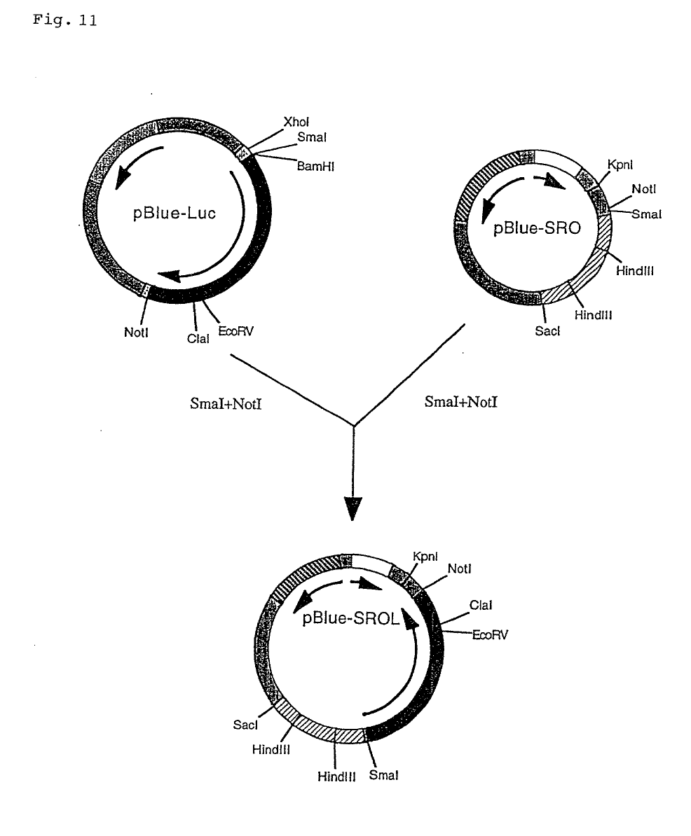

pBlue-SRO was digested with SmaI and NotI to obtain a DNA fragment (about4.2kb). Separately, pBlue-Luc was digested with NotI and SmaI, and a DNA fragment (about 1.8 kb) containing the luciferase gene was purified. These two fragments were ligated, and the competent cells of E. coli K-12 strain, XL1-Blue, were transformed with the ligation product. The obtained colonies were cultivated and isolated plasmids were analyzed. Thus, pBlue-SROL, in which the luciferase gene was inserted downstream to the SRα promoter, was constructed (FIG. 11).

(5) Construction of pSROL-3′SS(NB2)

5-1) Construction of p3′SS(-Xma I)

p3′SS (TOYOBO, STRATAGENE) was linearized by XmaI digestion, blunted with DNA Ligation Kit (TAKARA), and self-ligated. The competent cells of E. coli K-12 strain, XL1-Blue, were transformed with the resulting plasmid. The obtained colonies were cultivated, and isolated plasmids were analyzed to obtain p3′SS(-Xma I) with no XmaI site. The purpose of this treatment is to disrupt the Xma I site within the DNA derived from p3′SS for constructing the cDNA library later. (Being isoshizomers, both Xma I and SmaI recognize and cleave the sequence “CCCGGG”. To leave only the one sequence “CCCGGG” uniquely at the site where cDNA was inserted in the plasmid vector for constructing a cDNA library, the recognition site “CCCGGG” of SmaI was disrupted by mutating the sequence “CCCGGG” within the DNA derived from p3′SS into “CCCGGCCGGG” as described above.)

5-2) Construction of pSROL-3′SS(NB2)

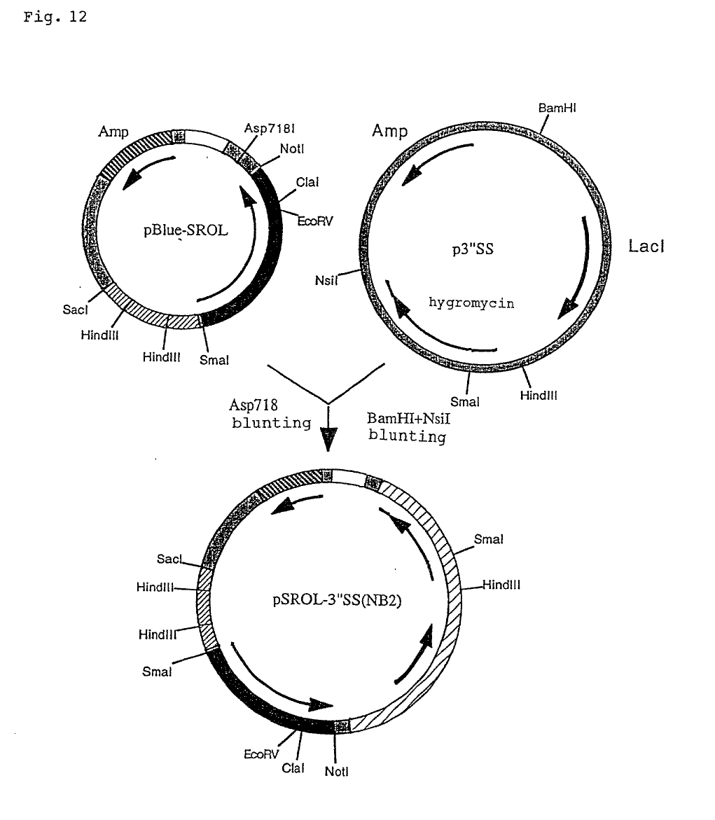

A DNA fragment (about 4.3 kb) generated by digesting p3′SS(-Xma I) with BamHI and NsiI was recovered by low-melting point agarose gel electrophoresis and purified with a tip for isolating and purifying nucleic acid, QIAGEN-tip 5 (FUNAKOSHI, QIAGEN Inc.). This fragment comprises units for expressing the hygromycin resistance gene and the Lac repressor. The hygromycin resistance gene is a selective marker used in gene transfer to animal cells, and the Lac repressor acts on Lac operator sequence. Separately, pBlue-SROL was digested with Asp718 and purified in the same manner as described above. These two fragments were blunted with a DNA Blunting Kit (TAKARA) then ligated with a DNA Ligation Kit (TAKARA). The ligation product was introduced into the competent cells of E. coli K-12 strain, XL1-Blue, to thereby obtain colonies resistant to ampicillin. The plasmids from the thus-obtained clones were analyzed, and pSROL-3′SS(NBl) and pSROL-3′SS(NB2) were obtained. In these two plasmids, the BamHI-NsiI fragment derived from p3′SS(-Xma I) was inserted in different directions. pSROL-3′SS(NB2) (FIG. 12), in which the direction of transcription from the three genes (the luciferase gene, the hygromycin gene, and the Lac repressor gene) is the same, was used in the experiments below.

### EXAMPLE 3

### Construction of cDNA Library for Cloning

(1) Preparation of poly(A)⁺ RNA

About 2×10⁸ MRC-5 SV1 TG1 cells (Riken Cell Bank) were cultivated for 5 hours after addition of 1×10⁻⁶ M dexamethasone (DEX). About 5 mg of RNA was extracted from the cells by the Acid Guanidium Thiocyanate Phenol Chloroform (AGPC) method (Chomczynski, P. and Sacchi, N. Anal. Biochem. 162, 156-159 (1987)). About 15 μg of poly(A)⁺ RNA was purified from 500 μg of the RNA using Oligotex-dT30 (TAKARA). This purification procedure was repeated twice.

(2) Preparation of Vector DNA

After being digested with NotI, pSROL-3′SS(NB2) was dephosphorylated at its ends by Bacterial Alkaline Phosphatase (BAP) (TAKARA) treatment then digested with SmaI. A 8.5 kb desired DNA fragment was recovered by low-melting point agarose gel electrophoresis.

(3) Synthesis of cDNA and Construction of Library

The process from the first-strand cDNA synthesis to ligation with a vector was performed in accordance with the “Method for Gene Library Construction” (Experimental Medicine: SUPPLEMENT, BIOMANUAL SERIES 2, YODOSHA, 79-94) using a ZAP-cDNA SYNTHESIS KIT (TOYOBO, STRATAGENE) as reagents as follows. M-MuLV Reverse Transcriptase synthesized 2.6 μg of first-strand cDNA using 10 μg of the poly(A)⁺ RNA described above as the template. The second-strand cDNA was then synthesized using E. coli RNase H and E. coli DNA polymerase. The resulting DNA was blunted with T4 DNA polymerase and digested with NotI. Low molecular weight DNAs were purified on a CHROMASPIN-400 column (TOYOBO, Clontech). Finally, 0.6 μg of double-stranded DNA was obtained. The thus-obtained cDNA was ligated using T4 DNA ligase into pSROL-3′SS(NB2) digested with SmaI and Not I ((2) described above). The reaction mixture was mixed with E. coli DH10B strain (ELECTRO MAX DH10B cell, GIBCO BRL) to introduce the cDNA into the cells using the CELL-PORATOR system (GIBCO BRL). It has been revealed that the library comprises 1.6×10⁶ colonies as a whole. Moreover, 90% of the clones contained the cDNA insert.

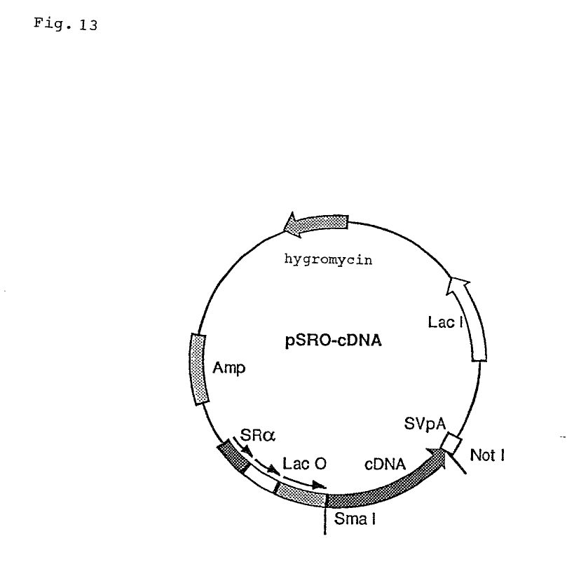

FIG. 13 shows the structure of the plasmid with cDNA constructed as described above. The plasmid can be used to incorporate cDNA of an antiinflammatory protein and transfected into animal cells to express the protein in the cells.

This vector can be used not only for transient expression of proteins coded by the cDNAs but also for obtaining stable (permanent) transfectants because it has the hygromycin resistence gene as a selective marker. When luciferase cDNA was used as an example of cDNA, a sufficient amount of protein (luciferase) was produced (expressed)

### EXAMPLE 4

### Construction of the Luciferase Expression Vector, pIL8p-Luc

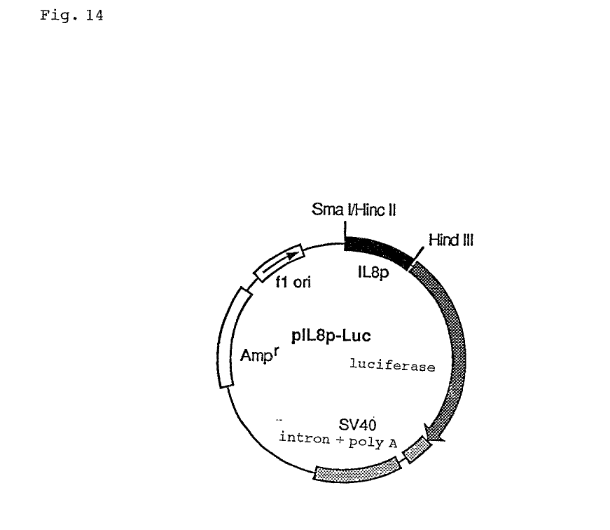

DNA comprising the human IL-8 (neutrophil chemotactic cytokine) promoter region (Matsushima, K. et al., J. Imm. 143, 1366-1371 (1989)) prepared by PCR using a genomic DNA of human VA-13 cells (Riken Cell Bank) was digested with HincII and HindIII to obtain a DNA fragment of −546 to +44 (IL8p). This fragment (IL8p) was inserted into pGL2-Promoter Vector (Promega) whose SV40 early promoter was removed with SmaI and HindIII, producing pIL8p-Luc. FIG. 14 shows the structure of this plasmid.

### EXAMPLE 5

### Cell Cultivation

MRC-5 SV1 TG1 cells (Riken Cell Bank), known as HGPRT-deficient cells, were cultivated in RITC 80-7 medium supplemented with 10% FCS. Dexamethasone dissolved in 100% ethanol was diluted with the same medium and added to the culture. Ethanol alone was added to the control group in the same concentration as ethanol added to the dexamethasone-treated group.

### EXAMPLE 6

### Quantitative Measurement of the Survival Rate of Cells

Cell survival was judged by quantitatively measuring the amount of the reduced product of Alamar Blue (KANTO KAGAKU), which was taken into cultured cells, in terms of fluorescence intensity or absorbance. This compound is reduced in the living cells and linked with the NADPH production system. The reduction product emits a characteristic color or fluorescence to be measured. After cells were cultivated in a 96-well plate, the medium was renewed, Alamar Blue was added, and the cells were incubated for 3 hours. The luminescence (fluorescence) at 590 nm was then measured by exciting the culture supernatants at 530 nm with a CytoFluor 2350 (MILLIPORE).

### EXAMPLE 7

### Establishment of HGPRT-Deficient Cell Clone that Stably Maintains the gpt Gene with the IL-8 Promoter

pREP9-IL8p-gpt-neo (FIG. 1), which had the gpt gene fused with the human IL-8 promoter and the neomycin resistance gene neo, was digested with ClaI and SphI to remove ori and EBNA-1 regions derived from the EB virus vector (pREP9) and to open the circular structure. MRC-5 SV1 TG1 cells deficient in HGPRT were transfected with the thus-obtained linearized DNA (IL8p-gpt-neo), which was made easy to integrate into the chromosome, and screened with 400 μg/ml G418. Twenty-one clones were isolated using cloning syringes. These clones were cultivated on a 12-well plate at a concentration of about 5×10⁴ cells/ml, treated with 1 μM dexamethasone for 3 hours, then cultivated with 1000 ng/ml 6-TG under stimulation by 20 to 100 U/ml (1 to 5 ng/ml) TNF-α.

In two of these clones (#17 and #21), cell death was induced by TNF-α stimulation in the presence of 6-TG and inhibited by treatment with dexamethasone.

Based upon the results above, a method quantitatively superior to the method for judging the effect of dexamethasone by observing cell survival under a microscope (e.g., a method for judging survival of cells by quantitatively measuring the amount of the reduction product of Alamar Blue (KANTO KAGAKU) which has been taken into cultured cells in terms of fluorescence intensity or absorbance) was tested. This compound is reduced in cells, linked with the NADPH production system, and emits characteristic color or fluorescence. The test was performed as follows.

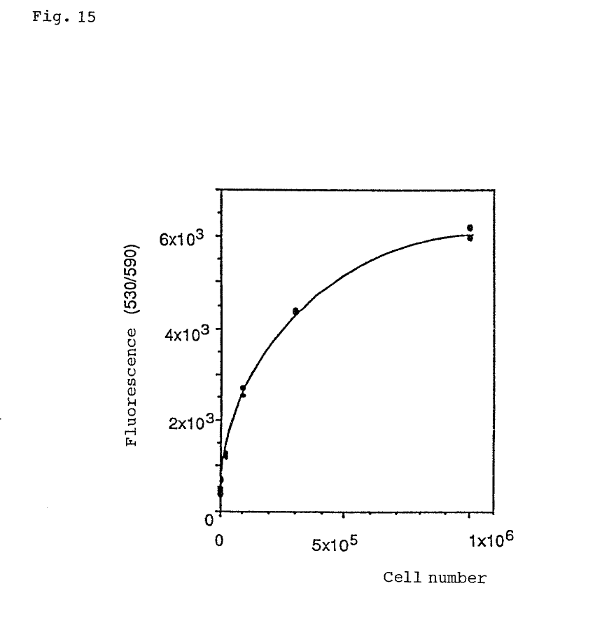

After 10⁶ MRC-5 SV1 TG1 cells, deficient in HGPRT, were cultivated in a 96-well plate, the medium was exchanged, Alamar Blue was added, and the cells were incubated for 3 hours. The luminescence (fluorescence) at 590 nm was then measured by exciting the culture supernatants at 530 nm with a CytoFluor 2350 (MILLIPORE) (FIG. 15).

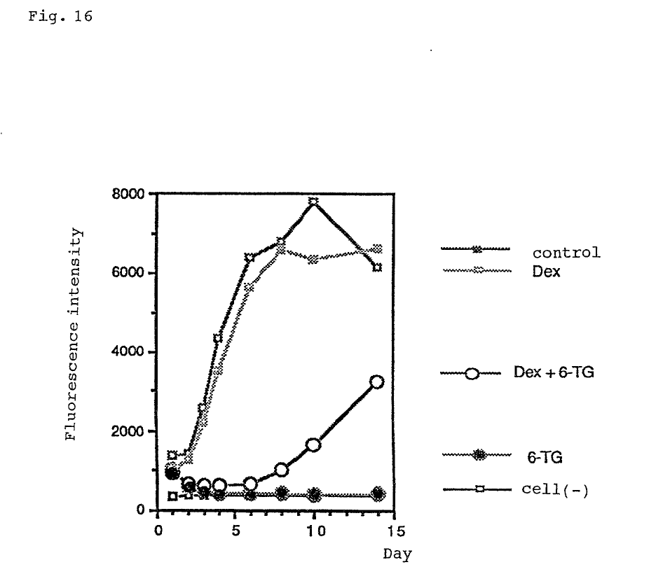

Based upon the results, 5×10³ cells of Clone #17 (IL8p-gpt-neo #17), which stably maintained IL8p-gpt-neo, were cultivated in a 96-well plate, and living cells were quantitatively measured using Alamar Blue by adding 1000 U/ml TNF- a in the presence of 1 μM dexamethasone and 1 μg/ml 6-TG. The results are shown in FIG. 16 and reveal that the cells of Clone #17 (IL8p-gpt-neo #17), which stably maintains IL8p-gpt-neo, all died when 6-TG was added and TNF- a stimulation applied, and that dexamethasone inhibited cell death and kept cells growing. Thus, the cell clone IL8p-gpt-neo#17, which dies if IL-8 promoter is activated by TNF stimulation but which is rescued from the cell death by treatment with the antiinflammatory steroid, has been developed by stably introducing the gpt gene with an IL-8 promoter into HGPRT-deficient cells. This cell clone can be used as a system for assessing cDNA encoding an antiinflammatory protein that inhibits the activation of the IL-8 promoter. Clone #17 (IL8p-gpt-neo #17), which stably maintains IL8p-gpt-neo, was deposited with the National Institute of Bioscience and Human-Technology, Agency of Industrial Science and Technology, Ministry of International Trade and Industry (1-1-3, Higashi, Tsukuba-shi, Ibaraki 305, Japan) on Oct. 9, 1996 (accession number FERM BP-5706) under the Budapest Treaty.

### EXAMPLE 8

### First Screening of cDNA Library (pSRO-cDNA library) using HGPRT-Deficient Cell Clone IP8p-gpt-neo #17 which Stably Maintains IL8p-gpt-neo

The first screening was performed by introducing the pSRO-cDNA library into IL8p-gpt-neo #17 cells (FIG. 16). Since the plasmids have the hygromycin resistance gene as the marker, only cells into which the plasmids are introduced survive by hygromycin selection. In contrast, the cells die if TNF-α and 6-TG are added to the culture medium. However, if the pSRO-cDNA library has a cDNA encoding protein that inhibits the activation of the IL-8 promoter (antiinflammatory protein), cells in which the cDNA is expressed must survive. The experiment was performed as follows.

250 μg of pSRO-cDNA library (size 1.6×10⁶) was introduced into IL8p-gpt-neo #17 cells (2.5×10⁷ cells; five 500 cm² trays) using Transfectam. Two days later, 100 μg/ml hygromycin was added. Five days thereafter, 20 mM IPTG was added, and the next day, 200 U/ml TNF and 1 μg/ml 6-TG were added to conduct screening. Consequently, several colonies per tray survived. Twenty-two clones were isolated on the twelfth day after addition of TNF and 6-TG and cultivated in the presence of hygromycin. Finally, cells of 16 strains were obtained.

### EXAMPLE 9

### Isolation of cDNAs Obtained in the First Screening

Genomic DNA was isolated from the 16 strains obtained in the first screening, and the pSRO-cDNA-library-derived cDNA insert introduced into the genomic DNA was extracted by PCR as described below.

1) Genomic DNA was prepared with a QIAamp Blood kit (QIAGEN).

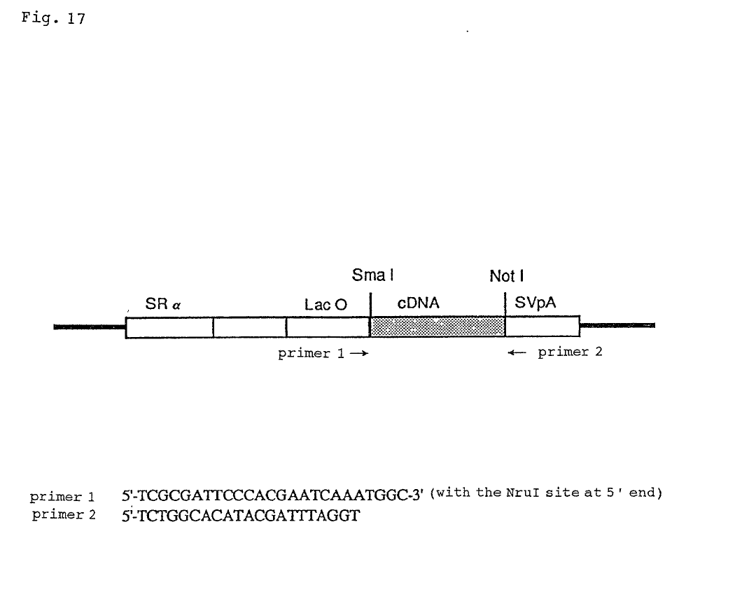

2) PCR was performed using the Ex Taq (TaKaRa) and Gene Amp PCR system 9600 (Perkin Elmer) for 30 cycles of incubation at 96° C. for 60 seconds, at 60° C. for 30 seconds, and at 72° C. for 30 seconds, followed by incubation at 72° C. for 6 minutes. Sequences immediately before the cDNA (a part of LacO sequence) and immediately after the NotI site (a part of SVPA sequence) in pSRO-cDNA (FIG. 13) were used as PCR primers (FIG. 17).

Agarose gel electrophoresis revealed that nine kinds of PCR products were obtained from genomic DNAs isolated from 5 out of the 16 strains described above.

Next, these PCR products were digested with Nru I and Not I, and ligated with the purified fragments generated by digesting pSROL-3′SS(NB2) with SmaI and NotI. The ligation product was introduced into competent cells of E. coli JM109 to obtain colonies resistant to ampicillin. The plasmids from the colonies were analyzed. Expression vector pSRO-cDNA (FIG. 13), which corresponds to nine kinds of cDNAs collected by PCR, was thus selected.

The nucleotide sequences of the nine kinds of pSRO-cDNA were analyzed. The second screening was performed as shown below.

### EXAMPLE 10

### Second Screening by Reporter Gene Method

The second screening was performed for cDNAs obtained in the first screening to confirm the inhibitory effect on the IL-8 promoter. The second screening was performed by the reporter gene method in which pIL8p-Luc (FIG. 14) was used as a luciferase expression vector having the IL-8 promoter.

If pSRO-cDNA, the expression vector of the cDNA obtained in the first screening (FIG. 13), and pIL8p-Luc are co-transfected at a ratio of 10:1, cells taking pIL8p-Luc also take the target cDNA expression vector into themselves (Analytical Biochemistry 188, 245-254 (1990)). Therefore, in this system, whether or not introducing the expression vector of the cDNA to be tested can inhibit the activation of the IL-8 promoter by TNF-α stimulation was examined. Furthermore, pSvβ (CLONTECH, 6178-1), the expression vector of β-galactosidase, was used as the internal reference for compensating the transfection efficiency of the expression vectors.

On the first day, 2×10⁵ cells of IL8p-gpt-neo #17 were cultivated on a six-well plate. On the second day, transfection was performed using Transfectam by adding 1 μg of cDNA to be tested (pSRO-cDNA, FIG. 13), 0.1 μg of IL8p-Luc (FIG. 14), and 0.2 μg of pSVβ (internal reference) per well. Three to four wells were used for every cDNA. On the third day, IPTG was added to 20 mM (final concentration). On the fourth day, 1 μM dexamethasone (final concentration) was added. After three hours, TNF was added to 100 U/ml (final concentration). After five hours, cells were collected with a cell lysis agent LCβ (TOYO INK), and cell lysate was prepared. The luciferase activity in the lysate was then measured with PicaGene (TOYO INK). β-galactocidase activity was also measured with Galacto-Ligt (TROPIX) after endogenous galactocidase activity was eliminated by heating the cell lysate at 48° C. for 50 minutes. pSRO without cDNA was used as the control of pSRO-cDNA. The data were shown in terms of luciferase activity compensated by β-galactocidase activity (internal reference).

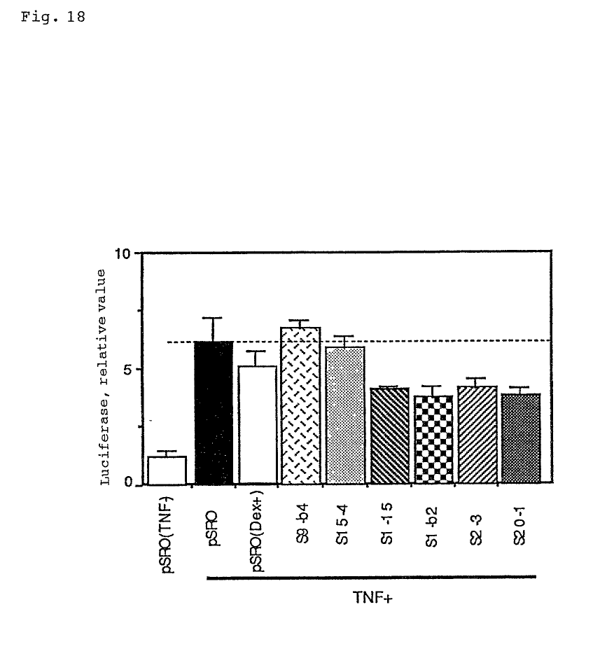

Consequently, TNF induced strong luciferase activity in the cells into which pSRO without cDNA was introduced, whereas treatment with dexamethasone inhibited the activity (FIG. 18). In the test cDNAs, S1-15, S1-b2, S2-3, and S20-1 showed inhibitory activity on the IL8 promoter, but S9-b4 and S15-4 showed no inhibitory activity (FIG. 18).

Industrial Applicability

The present invention provides a method for detecting an inhibitory effect of a test substance on intracellular signal transduction and a method for isolating a gene encoding a protein that inhibits intracellular signal transduction. The present invention makes it possible to easily detect whether a specific test substance has the inhibitory effect on intracellular signal transduction and to efficiently screen cDNA encoding a protein that inhibits intracellular signal transduction from a cDNA library composed of the mixture of a million or more cDNAs.

1. A vector holding a gene capable of inducing cell death under specific conditions and linked downstream to a promoter region that functions in response to specific extracellular stimulation.

2. The vector of claim 1, wherein said extracellular stimulation is stimulation by cytokine.

3. The vector of claim 1, wherein said extracellular stimulation is stimulation by tumor necrosis factor (TNF).

4. The vector of claim 3, wherein said promoter region functioning in response to specific extracellular stimulation is a promoter region of interleukin 8 gene.

5. The vector of any one of claims 1 to 4, wherein said gene inducing cell death under specific conditions is xanthine-guanine-phosphoribosyltransferase (gpt) gene.

6. A host cell transformed with the vector of any one of claims 1 to 5.

7 The host cell of claim 6, wherein said cell cannot produce hypoxanthine-guanine-phosphoribosyltransferase (HGPRT), and said gene inducing cell death under specific conditions in the vector to be inserted is xanthine-guanine-phosphoribosyltransferase.

8. A method for detecting an inhibitory effect of a test substance on intracellular signal transduction, wherein the method comprises,
(a) introducing the test substance into or allowing the substance to act on the cells of claim 6,
(b) testing whether the cells obtained in (a) that the test substance is introduced into or acts on are alive or not when the specific extracellular stimulation which causes said specific intracellular signal transduction under the specific conditions is added to the cells.

9. A method for detecting an inhibitory effect of a test substance on intracellular signal transduction, wherein the method comprises,
(a) introducing the test substance into or allowing the substance to act on the cells of claim 7,
(b) testing whether the cells that the test substance is introduced into or acts on obtained in (a) are alive or not when the specific extracellular stimulation which causes said specific intracellular signal transduction is added to the cells in the presence of 6-thioguanine.

10. The method of claim 8 or 9, wherein said test substance is a gene.

11. The method of claim 8 or 9, wherein said test substance is a low molecular weight compound.

12. A method for isolating a gene encoding a protein that inhibits specific intracellular signal transduction, wherein the method comprises,
(a) introducing a gene library into the cells of claim 6,
(b) screening living cells after the specific extracellular stimulation which causes said specific intracellular signal transduction is added under the specific conditions to the cells that the gene library is introduced into obtained in (a),
(c) isolating the gene introduced into said cells from the cells screened in (b).

13. A method for isolating a gene encoding a protein that inhibits specific intracellular signal transduction, wherein the method comprises,
(a) introducing a gene library into the cells of claim 7,
(b) screening living cells after the specific extracellular stimulation which causes said specific intracellular signal transduction is added in the presence of 6-thioguanine to the cells that the gene library is introduced into obtained in (a),
(c) isolating the gene in said cells from the cells screened in (b).

14. The method of any one of claims 8 to 13, wherein said extracellular stimulation is stimulation by cytokine.

15. The method of any one of claims 8 to 13, wherein said extracellular stimulation is stimulation by tumor necrosis factor (TNF).

16. The method of claim 15, wherein said promoter region functioning in response to specific extracellular stimulation is a promoter region of interleukin 8 gene.

17. The method of any one of claims 8 to 13, wherein said vector introduced into the cells of claim 6 or 7 is the vector of claim 4.

18. A method for detecting an inhibitory effect of a test gene on intracellular signal transduction, wherein the method comprises,
(a) introducing into host cells a vector comprising a test gene that can be expressed in the host cells and a vector having a reporter gene downstream to a promoter region functioning in response to specific extracellular stimulation,
(b) applying specific extracellular stimulation to the host cells obtained in (a) into which the vector is introduced and detecting the activity of a reporter gene product.

19. A method for detecting an inhibitory effect of a low molecular weight compound to be tested for intracellular signal transduction, wherein the method comprises,
(a) introducing into host cells a vector having a reporter gene downstream to a promoter region functioning in response to specific extracellular stimulation,
(b) allowing a low molecular weight compound to be tested to act on the host cells into which the vector obtained in (a) is introduced and detecting the activity of the reporter gene product.

20. A method for isolating a gene encoding a protein that inhibits specific intracellular signal transduction, wherein the method comprises,
(a) introducing into host cells a gene library that can be expressed in the host cells and a vector having a reporter gene downstream to a promoter region functioning in response to specific extracellular stimulation,
(b) applying specific extracellular stimulation to the host cells into which the vector in (a) is introduced, detecting the activity of the reporter gene product, and selecting cells in which said activity decreases,
(c) isolating a gene introduced into said cells from the cells screened in (b).

21. The method of any one of claims 18 to 20, wherein said reporter gene is the luciferase gene.

22. The method of any one of claims 18 to 21, wherein said extracellular stimulation is stimulation by cytokine.

23. The method of any one of claims 18 to 21, wherein said extracellular stimulation is stimulation by tumor necrosis factor (TNF).

24. The method of claim 23, wherein said promoter region functioning in response to specific extracellular stimulation is a promoter region of the interleukin 8 gene.

25. A host cell transformed with a vector comprising a test gene that can be expressed in the host cells and a vector having a reporter gene downstream to a promoter region functioning in response to specific extracellular stimulation.

26. The host cells of claim 25, wherein said extracellular stimulation is stimulation by cytokine.

27. The host cells of claim 25, wherein said extracellular stimulation is stimulation by tumor necrosis factor (TNF).

28. The host cells of claim 27, wherein said promoter region functioning in response to specific extracellular stimulation is a promoter region of interleukin 8 gene.

29. The host cells of any one of claims 25 to 28, wherein said reporter gene is luciferase gene.


SEQUENCE LISTING (4 sequences)

SEQ ID NO: 1 - DNA, 29 bp, Artificial Sequence
  Artificially synthesized primer sequence
ataagcttttcacatgagcgaaaaataca

SEQ ID NO: 2 - DNA, 29 bp, Artificial Sequence
  Artificially synthesized primer sequence
atggatccctattgtaacccgcctgaagt

SEQ ID NO: 3 - DNA, 38 bp, Artificial Sequence
  Artificially synthesized primer sequence
atgtctcgagaattcagtaacccaggcattattttatc

SEQ ID NO: 4 - DNA, 30 bp, Artificial Sequence
  Artificially synthesized primer sequence
ttgtcctagaagcttgtgtgctctgctgtc